(19) United States
(12) Patent Application Publication
(10) Pub. No.: US 2011/0195503 A1
(43) Pub. Date: Aug. 11, 2011

(54) GOLD NANOPARTICLE, A COMPOSITION AND A METHOD TO PERPETUATE STEMNESS THEREOF

(75) Inventors: Satish Mahadeo Totey, Bangalore (IN); Kaushik Dilip Deb, Bangalore (IN); Kavitha Sivasubramaniyan, Bangalore (IN)

(73) Assignee: STEMPEUTICS RESEARCH PRIVATE LIMITED., Bangalore (IN)

(21) Appl. No.: 13/087,243

(22) Filed: Apr. 14, 2011

(30) Foreign Application Priority Data
Oct. 24, 2008 (IN) 02601/CHE/2008

(51) Int. Cl. C12N 5/071

(52) U.S. Cl. 435/405; 435/325; 435/404

(57) ABSTRACT

The present invention relates to gold nanoparticle (GNP) and a method to perpetuate stemness of stem cells comprising step of growing the stem cells in presence of gold particle or Swarna Bhasma. It also relates to compositions for perpetuating stemness of stem cells.

### REFERENCE TO RELATED APPLICATIONS

This application is a National Stage entry from a PCT International application number PCT/IN2008/000816 having International Filing Date of 5 Dec. 2008 and Priority Date of 24 Oct. 2008. This application claims the benefit of the PCT application, said International Filing Date and said Priority Date under 35 USC sections 119, 120, and 365.

### BACKGROUND

The present invention relates to field of stem cell research. It relates to the use of Swarna bhasma or gold nanoparticles in perpetuating stemness of stem cells.

Stem cells are associated with tissue formation during embryogenesis and in their repair and maintenance during injuries and aging in the adults. These cells are unique in their capacity to 1) self-renewal, making more copies of itself and 2) retain potential to differentiate into multiple (multipotent) or all the germ lineages, pluripotency (1, 2, 3). These properties which distinguish them from other cell types are collectively defined as the “stemness” of these cells (4, 5). Embryonic stem cells (ESC) are derived from the inner cell mass of a peri-implantation embryo and are known to be pluripotent (Deb et al., Rej res, 2008). During subsequent development towards formation of a complete organism, the cells of the embryo chronologically progress through proliferation, lineage commitment, lineage progression, lineage expression, cellular inhibition and regulated apoptosis resulting in the formation and maintenance of the differentiated cells, tissues, and organs in an individual (6, 7). Although most cells advance through this sequence during development, a few cells leave the developmental continuum to become reserve precursor cells in the adult tissue (7, 8). There are two categories of these precursor cells namely lineage-committed or multipotent, somatic stem cells and lineage-uncommitted pluripotent stem cells known as the embryonic like stem cells (ELSCs). These reserve precursor cells incessantly maintain and repair tissues and organs throughout the life span of the organism (9, 10, 11). It is also believed that owing to their pluripotent capacity, ELSCs are the most potent tissue resident stem cells, and activation of this population may lead to the best possible tissue regeneration (12, 13). Moreover, it has been reported that the tissue resident ELSCs, for example of muscle tissue, can also get mobilized to the peripheral blood after trauma (13). Further, Kucia et al (14) demonstrated the mobilization of human adult-derived ELSCs into the circulatory system after stroke (15).

The use of gold and gold compounds for therapy in ancient Greek, Arabic, Chinese and Indian systems of medicine and in modern medicine has been reviewed and discussed at length in literature (16-23). In the 5000 year old Indian traditional medicine, Swarna bhasma (SB) meaning gold ash, has been extensively used for treating various clinical disorders like bronchial asthma, rheumatoid arthritis, diabetes mellitus, nervous diseases, etc. (Kean et al., 1985; Zhao and Ning 2001).

Swarna Bhasma, a traditional mixture was recently physiochemically characterized and studied for anti-arthritic activities (Brown CL et al, gold bulletin 2007). Brown et al., (2007) showed that the gold particles in these SB preparations were zerovalent and of average 27±3 nm in size, have properties similar to that of modern day nanoparticles. In an earlier study, employing X-ray diffraction (XRD) analysis, Brown CL et al (2007) showed that the SB preparations were free of organic compounds and mainly consisted of standard gold metal (Auo). Their results also indicated that the average size of the particles were about 57 nm and had a globular morphology.

The most active area of research in gold based pharmaceuticals at present is in its application as anti-tumor agents (22, 25-29). Several other metals are also used in the Indian traditional medicine after converting them into oxides or sulfides mainly to eliminate their reactivity. Metallic gold (AuO), which is practically non-reactive or inert anyway, only needs to be sufficiently small particles, enabling them to circulate in the system and exert their effects much longer than ordinary medicines can. SB is given orally to patients, usually mixed with honey. It is believed that, in this way the gold particles which are often of nano size, and expected to have attained nanoparticulate properties, get absorbed through sublingual route directly into blood stream like a homeopathic drug (Brown CL et al., 2007 Gold bulletin). Another recent study by Hillyer and Albrecht (2006) demonstrated that gold nanoparticles smaller than 58 nm in size is absorbed in the small intestine and reach various organs through blood. In the recent years, nanoparticles of gold have attracted tremendous attention for their applications in medicine (25, 26, 30).

Human embryonic stem cells (hESCs) recapitulate organogenesis when they differentiate into tissues of various lineages. They have been widely used as suitable tools to study developmental toxicity and as a model source for understanding the molecular mechanisms of pluripotency (Sivasubramaniyan et al., 2008). The in vitro properties of the hESC make them attractive targets for research and therapy (Deb and Sarda, JTM, 2008). They provide an unlimited source of any cell type owing to their capacity to sustain pluripotency. However, the greatest draw back of maintaining hESCs in culture is their tendency to differentiate spontaneously (31). In the most commonly practiced hESCs cultures, which involve FGF-2 supplementation to sustain pluripotency, the hESCs need to be passaged on every third-fourth day to maintain their characteristics which is expensive and time consuming (32). The hESCs spontaneously differentiate and tend to loose their proliferation capacity in culture from day 4 onwards, thereby loosing their “stemness” over time. This presents an unprecedented in vitro system to test the effect of growth factors and compounds on maintenance of pluripotency and self renewal.

Several groups have also reported about biological additives (growth factors and various pathway inhibitors) that can improve hESC culture conditions and reduced spontaneous differentiation of hESCs in cultures (references Ludwig et al., 2006 (a) and (b) nat methods and nat biotech).

The present invention overcomes the problem associated with the prior art mentioned above.

### OBJECTS OF THE INVENTION

The main object of the present invention is to develop a method to perpetuate stemness of stem cells using gold particles or Swarna bhasma.

Another object of the present invention is to obtain gold nanoparticle (GNP) at concentration of about 0.1 μg/ml to about 20 μg/ml for perpetuating stemness of stem cells.

Yet another object of the present invention is to obtain a composition comprising fibroblast growth factor along with gold particles or Swarna bhasma.

Still another object of the present invention is to obtain a medium for perpetuating stemness of stem cells, said medium comprising components of ES media along with gold particle or Swarna Bhasma.

Still another object of the present invention is to obtain a composition comprising Swarna bhasma and gold particles.

### SUMMARY

Accordingly, the present invention relates to gold nanoparticle (GNP) at concentration of about 0.1 μg/ml to about 20 μg/ml for perpetuating stemness of stem cells; a method to perpetuate stemness of stem cells comprising step of growing the stem cells in presence of gold particle or Swarna Bhasma; a composition for enhancing pluripotency of stem cells, said composition comprising Fibroblast Growth Factor along with gold particles or Swarna Bhasma; a medium for perpetuating stemness of stem cells, said medium comprising components of ES media along with gold particle or Swarna Bhasma; and a composition for perpetuating stemness of stem cells, said composition comprising Swarna Bhasma and gold particles, preferably gold nanoparticles.

### BRIEF DESCRIPTION OF THE DRAWINGS

FIG. 1a: Graph showing size distribution by volume of the SB preparation

FIG. 1b: Graph showing size distribution by volume of the GNP preparation

FIG. 1c: Transmission Electron Microscopy picture of GNP

FIG. 1d: Transmission Electron Microscopy picture of SB

FIG. 2a: Showing the effect of different doses of SB preparation on HUES9 grown in normal hESC media, by MTT assay.

FIG. 2b: Showing the effect of different doses of GNP on proliferation and pluripotency of hESCs.

FIG. 2c: Karyotype of HUES9 P15 grown in the presence of SB

FIG. 3a: QRT-PCR for ABCG2 in day 8 SB treated samples

FIG. 3b: QRT-PCR for ABCG2, Oct4 and Nanog in day 5 GNP treated samples

FIG. 3c: QRT-PCR for ABCG2, Oct4 and Nanog in day 5 SB treated samples

FIG. 3d: QRT-PCR for ABCG2, Oct4 and Nanog in day 8 GNP treated samples

FIG. 4a: RT-PCR analysis for the expression of: 1) pluripotency markers (OCT4 and NANOG); and 2) lineage markers a) ectoderm (β III Tubulin, Nestin); b) endoderm (GATA4, AFP); and c) mesoderm (Brachury, BMP2) in HUES7 & 9 cells grown in the absence (hES-C), presence of gold bhasma (hES-SB) and presence of gold nanoparticles (hES-GNP) and in EBs formed from HUES9 grown in the absence and presence of Au bhasma. Day 5 & 8 hES-C and hES-GNP showed the presence of most of the lineage markers but Day 5 and Day 8 hES-SB showed the absence of all other lineage markers except ectoderm genes. The EBs formed from hES-C, hES-SB and hES-GNP showed expression for all the lineage markers.

FIG. 4b: Immunolocalization of Oct4 and SSEA4 in HUES9. Panels (a) and (c) show the localization of Oct4 and SSEA4 in control respectively (b) and (d) show the localization of Oct4 and SSEA4 in presence of SB respectively, (c) and (f) show the localization of Oct4 and SSEA4 in presence of GNP respectively.

FIG. 5: Graphs showing cell proliferation after SB (1 ug/ml) and GNP (10 ug/ml) supplementation.

FIG. 6: Phase contrast pictures of Day 4 HUES-9 colonies and embryoid bodies (EBs). Panel (a) shows control HUES9 colonies. Panels (b) and (c) show morphology of undifferentiated HUES9 colonies growing on MEF in the presence of SB and GNP respectively. Panel (d) shows normal day 5 EBs. Panels (e) and (f) shows morphologies of Day 5 EBs induced from HUES9 grown in the presence of SB and GNP, respectively.

FIG. 7: Semiquantitative RT-PCR analysis of the expression of FGFR1 and IGF2 in day 8 HUES9 cells grown in the absence and presence of gold bhasma.

FIG. 8: Differential uptake of SB and GNP by embryonic and fibroblast niche cells.

FIG. 9: FACS analyses of the expression of Tra 1-60 in day 8 HUES9 cells grown in the absence and presence of SB or GNP plus FGF2.

### DETAILED DESCRIPTION

The present invention relates to gold nanoparticle (GNP) at concentration of about 0.1 μg/ml to about 20 μg/ml for perpetuating stemness of stem cells.

In another embodiment of the present invention, the concentration of GNP is preferably about 10 μg/ml.

In yet another embodiment of the present invention, the GNP has diameter of about 15 nm to about 16.5 nm.

In still another embodiment of the present invention, the stem cells are lineage-uncommitted pluripotent stem cells.

The present invention relates to a method to perpetuate stemness of stem cells comprising step of growing the stem cells in presence of gold particle or Swarna Bhasma.

In still another embodiment of the present invention, the method perpetuates stemness by enhancing proliferation, self-renewal and pluripotency and reducing spontaneous differentiation of the stem cells.

In still another embodiment of the present invention, the gold particle is gold nanoparticle (GNP).

In still another embodiment of the present invention, the gold nanoparticle (GNP) is present at a concentration of about 0.1 μg/ml to about 20 μg/ml, preferably about 10 μg/ml. In still another embodiment of the present invention, the Swarna bhasma is present at a concentration of about 0.1 μg/ml to about 2 μg/ml, preferably about 1 μg/ml.

In still another embodiment of the present invention, the stem cells are lineage-uncommitted pluripotent stem cells.

The present invention also relates to a composition for enhancing pluripotency of stem cells, said composition comprising Fibroblast Growth Factor along with gold particles or Swarna Bhasma.

In still another embodiment of the present invention, the gold particle is gold nanoparticle (GNP).

In still another embodiment of the present invention, the GNP is present at a concentration of about 0.1 μg/ml to about 20 μg/ml, preferably about 10 μg/ml.

In still another embodiment of the present invention, the GNP has diameter of about 15 nm to about 16.5 nm.

In still another embodiment of the present invention, the Swarna bhasma is present at a concentration of about 0.1 μg/ml to about 2 μg/ml, preferably about 1 μg/ml.

In still another embodiment of the present invention, the Fibroblast Growth Factor is a basic fibroblast growth factor or FGF2.

In still another embodiment of the present invention, the FGF2 is present at a concentration of about 2 ng/ml to about 40 ng/ml, preferably about 4 ng/ml.

In still another embodiment of the present invention, the stem cells are lineage-uncommitted pluripotent stem cells.

The present invention also relates to a medium for perpetuating stemness of stem cells, said medium comprising components of ES media along with gold particle or Swarna Bhasma.

In still another embodiment of the present invention, the gold particle is gold nanoparticle (GNP).

In still another embodiment of the present invention, the gold nanoparticle (GNP) is present at concentration of about 0.1 μg/ml to about 20 μg/ml, preferably about 10 μg/ml.

In still another embodiment of the present invention, the GNP has diameter of about 15 nm to about 16.5 nm, preferably about 15.59 nm.

In still another embodiment of the present invention, the Swarna bhasma is present at a concentration of about 0.1 μg/ml to about 2 μg/ml, preferably about 1 μg/ml.

In still another embodiment of the present invention, the stem cells are lineage-uncommitted pluripotent stem cells.

In still another embodiment of the present invention, the components of ES media include 80% Knockout DMEM, 20% Knockout serum replacer, L-glutamine at concentration of about 2 mM, 1% nonessential aminoacids, β-mercaptoethanol at concentration of about 0.1 mM, human FGF2 at concentration of about 4 ng/ml and penicillin streptomycin at concentration of about 50 U/ml.

The present invention also relates to a composition for perpetuating stemness of stem cells, said composition comprising Swarna Bhasma or gold particles, preferably gold nanoparticles.

The present invention relates to gold particulate preparation on improvement of the stem cell proliferation, self renewal and pluripotency. The gold particulate preparation, traditionally known as Swarna bhasma (SB) was adapted and prepared by the methods described in traditional Indian medicinal literature.

Trace element analyses of the SB preparations by inductively coupled plasma-optical emission spectrometry (ICPOES) showed no statistically significant variation in the trace elements compositions, measured in parts per million (ppm), across samples obtained from different batches. The presence of mercury and lead less than the minimal detection levels in the aforesaid preparations indicated a high quality product with lesser toxicity and better potential acceptability as a medicine.

With Dynamic light scattering studies, it was observed that more than 70% of the particles were of approximately 41 nm in size. Since, gold in the SB was found to be smaller than 58 nm in size, it is envisioned that they reach the tissues through blood even after oral administration.

In the present invention, the hESC cultures were supplemented with SB in presence and absence of FGF-2 to determine its effect on pluripotency. We found that SB supplemented at a concentration of 1 ug/ml supported increased cell proliferation and also increased the expression of pluripotency markers like ABCG2, as compared to the control. This dose also reduced spontaneous differentiation of the ESCs till day 8, without compromising their ability to give rise to cells of the three germ layer lineages. However, the SB particles exhibited their best effect up to day 8 as the cells showed complete silencing for most lineage markers.

Since SB is predominantly comprised of metallic inert gold (Au^{o}) nanoparticles, the role of synthesized colloidal gold (Au^{o}) nanoparticles (GNP) on the stemness of the cells was assessed. It was found that the expression of pluripotency genes like ABCG2 were upregulated significantly over the control. FIG. 8 provides the uptake of GNP's and SB by hES cells.

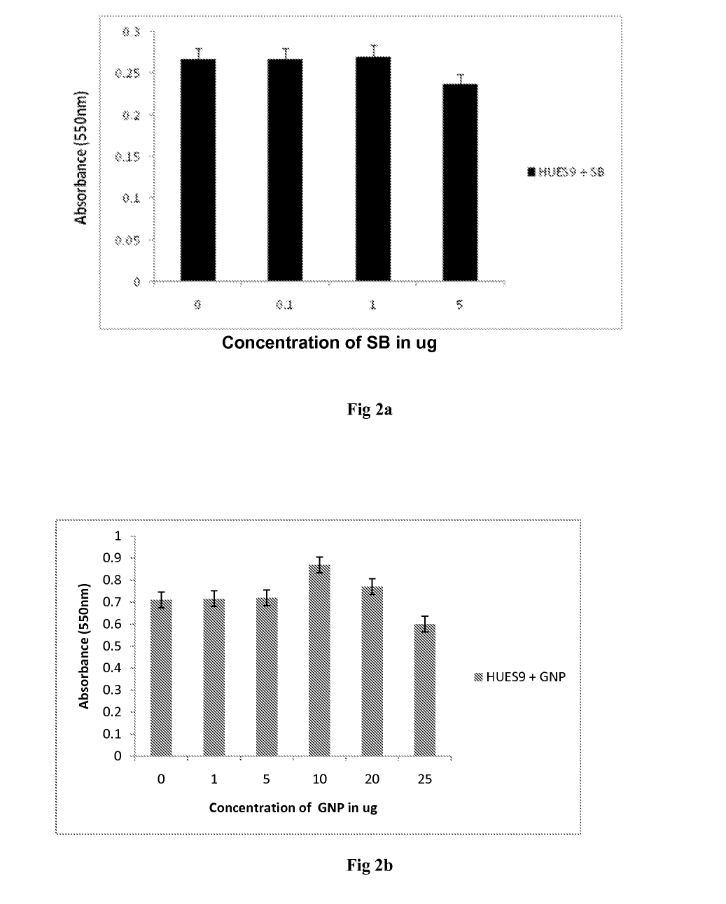

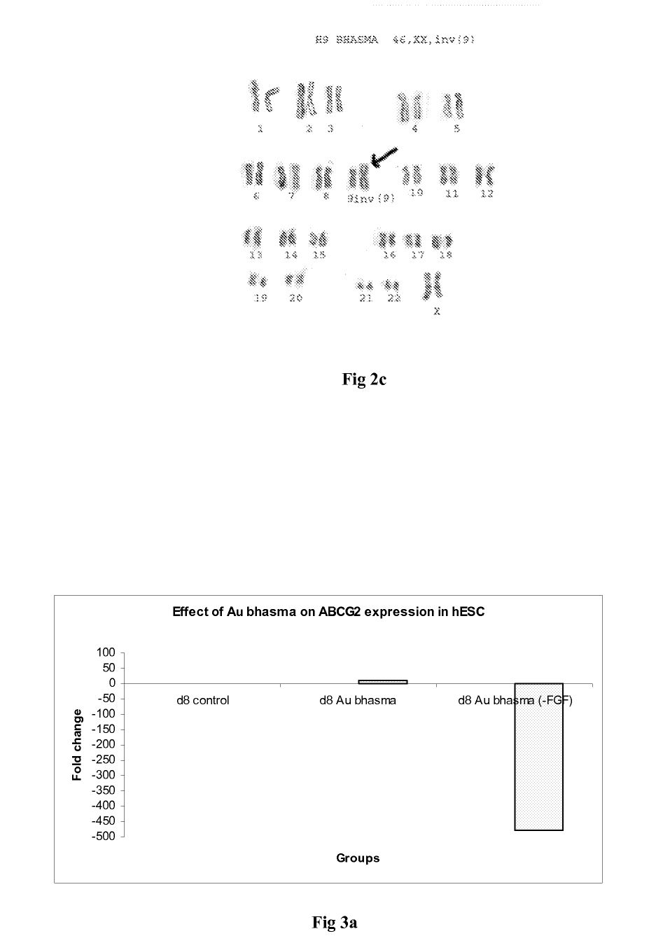

Intriguingly, this study also indicates that gold supplementation to the hESC culture media along with FGF-2 helps in improving the fidelity of the cultures and in sustaining prolonged pluripotency. Human ESC cultures are known to be susceptible to karyotypic abnormalities like trisomy 12 and/or 17 due to selection pressure during cultures over several passages. Therefore, it was imperative to confirm that the GNPs and the SB preparations did not negotiate the pluripotency and chromosomal stability of the ESCs even upto 15 passages (FIG. 2c).

Further, the present invention proves that gold brings about this effect by improving the niche population. The pool of niche cells in hESC is known to secrete IGF-2 and express FGFR1. The secreted IGF-2 in turn was shown to facilitate maintainance of pluripotency in hESCs. Likewise, an upregulation in the expression of FGFR1 and IGF-2 was detected. Further, an increase in the proliferation of the total population of cells in the SB treated culture plates from day 4 onwards was observed. These results implicate that SB preparations improve stemness by inducing the expression of IGF-2 in the niche cells without altering the ratio of niche cells in an ESC microenvironment. It appears obvious that due to an overall increase in the niche cell pool in the cultures, the total amount of secreted IGF-2 in the culture media is proportionately up-modulated.

Both SB and GNP when used in combination with FGF-2, significantly upregulated the expressions of pluripotency markers by day 5 and hence it is apparent that the gold nanoparticles in SB or GNP are the main players orchestrating this effect.

Another important aspect of any culture media supplements is their interference in the freeze-thaw efficiencies of the cells. Several media components are known to selectively improve or affect the freeze-thawing efficiencies of cells. Our preliminary studies indicate no adverse effect on the efficacy of the cells to revive after being cultured continuously in presence of SB or GNP over several passages. These findings strongly indicate that gold nanoparticles or SB at its determined dose can be supplemented in hESC cultures to improve hESC pluripotency. Their potential to alleviate the proliferation, pluripotency and self renewal in ESCs suggest that they could possibly bring about the same effects in vivo in the tissue resident pluripotent population of ELSCs. The study therefore provides an in vitro evidence for the function of gold preparations in enhancing rejuvenation and regeneration through hESC.

The invention is further elaborated with the help of following examples. However, these examples should not be construed to limit the scope of invention.

### Example 1

### Human Embryonic Stem Cell (hESC) Cultures and In Vitro Maintenance

hESC line HUES9 and HUES7 were obtained from Douglas Melton at Harvard. University. They were grown on mitomycin-C-treated CF1 mouse derived embryonic feeder (MEFs) cells at 37° C., 5% CO2 in the ES media which consists of 80% KO DMEM, 20% Knockout serum replacement, 2 mM L-Glutamine, 1% nonessential amino acid solution, 0.1 mM β-mercaptoethanol, 4 ng/ml human FGF2 and 50 U/ml pencillin-streptomycin (all from Invitrogen, CA, USA). The cells were passaged every 4 days to maintain pluripotency and self renewal.

Feeder-free undifferentiated HuES9 human ES cells were maintained on Matrigel (1:15 dilution, Sigma) coated dishes in conditioned medium containing knockout DMEM/20% serum replacement, 2 mM L-G utamine, 1% nonessential amino acid solution, 0.1 mM β-mercaptoethanol and 50 U/ml pencillin-streptomycin (all from Invitrogen, CA, USA). Conditioned medium was obtained by culturing mouse embryonic fibroblast (MEF) cells with HuES9 media. The medium was collected at 24 h intervals, filter sterilized and further supplemented with 8 ng/ml bFGF for HuES9 cell culture.

### Example 2

Swarna Bhasma (SB) preparation: One part of gold and eight parts of mercury were taken together in a mortar and amalgam was prepared by continuous trituration for 3 days. To this, sixteen parts of sulfur was added and triturated till homogenous mixture is obtained. This mixture was then taken in a mud smeared beer bottle and kept in the furnace in a specially designed vessel called “valuka yantra” (a metal bucket filled with coarse and uniform gravels of sand). Continuous and controlled heat was given for about 6-8 hrs. The bottle in the furnace was left for self cooling. The bottle was cut open in the middle and product from the bottom of the bottle was collected. The product from the bottom of the bottle was mixed well with equal parts of white arsenic (As₂O₃) and impregnated with juice of Ocimum sanctum. Now the contents were taken in an earthen crucible. The crucible was covered by placing another inverted earthen crucible on top of it and sealed by three layers of cotton cloth and wet clay. The assembly was heated for 8 h using cow dung cakes (eight cakes) in a pit. The maximum temperature during heating was ˜900° C. This was opened on the next day and product was collected. This was again mixed with half part of white arsenic and again impregnated with juice of Ocimum sanctum. Then it was kept in a crucible and ignited. This procedure was repeated for 8-10 times or till it attained proper qualities of final product. This product was repeatedly made over a number of times and analyzed for particle size and consistency in trace element compositions and batch variations.

Gold nanoparticle (GNP) preparation: To prepare the GNPs, 20 ml of 1 mM auric chloride was heated to boiling in an Erlenmeyer flask with stirring. 2 ml of a 1% solution of trisodium citrate was added slowly to this boiling solution and heating was continued for about 20 min. The citrate reduces auric chloride to gold nanosuspension. The completion of the reaction was detected by the formation of deep red suspension. This was analyzed for particle size subsequently.

### Example 3

Trace element analysis of SB preparations: Concentrations of minor and trace elements (As, Cd, Hg and Pb) in the swarna bhasma (SB) were analyzed by the inductively coupled plasma-optical emission spectrometry (ICPOES) method using Perkin Elmer Optima 5300DV. 10 mg of the bhasma was dissolved in 0.5 ml of Nitric acid. This was made up to 10 ml using deionised water to make a concentration of 10 mg/ml and was used for ICP-OES studies. An equivalent concentration of nitric acid was used as blank. Readings were made in axial mode and trace element concentration was evaluated from the standard calibration curve. To check if the compositions of the trace elements vary with the batches of SB preparations, trace element analyses for three different sample preparations SB1, SB2 and SB3 was done.

Trace element analysis using Optical Emission Spectroscopy with Inductively Coupled Plasma of the SB preparations showed detectable amounts of arsenic (29.27±0.67 ppm) and cadmium (0.636±0.02 ppm), while the level of mercury and lead were found to be below minimum detection levels (Table 1).

TABLE 1
Trace element analysis of the SB samples by inductively
coupled plasma- optial emission spectometry (ICPOES).

| | Element | | | |
|---|---|---|---|---|
| | Arsenic | Cadmium | Mercury | Lead |
| Sample code | (ppm) | (ppm) | (ppm) | (ppm) |
| SB1 | 29.70 | 0.622 | <MDL | <MDL |
| SB2 | 29.61 | 0.625 | <MDL | <MDL |
| SB3 | 28.5 | 0.66 | <MDL | <MDL |
| Average ± SD | 29.27 ± 0.67 | 0.636 ± 0.02 | | |

<MDL- less than the minimum detection limit of the
instrument. i.e., for mercury 3.38 ppm and for lead 1.533 ppm

Particle size analysis of SB and GNP: The particle size of the SB and GNP was analyzed using Nano ZS (Malvern Instruments Limited, UK). Dynamic light scattering studies were performed at 25° C. with the above equipment fitted with a He—Ne 4.0 mW (633 nm) laser. This enables the determination of the diffusion coefficient of the nanoparticles in solution and thereby calculates the hydrodynamic diameter (d·nm) of the particle using the Stokes-Einstein equation. Calculations were done automatically by the Dispersion Technology Software (v 5.00) of Malvern Instruments Ltd. UK.

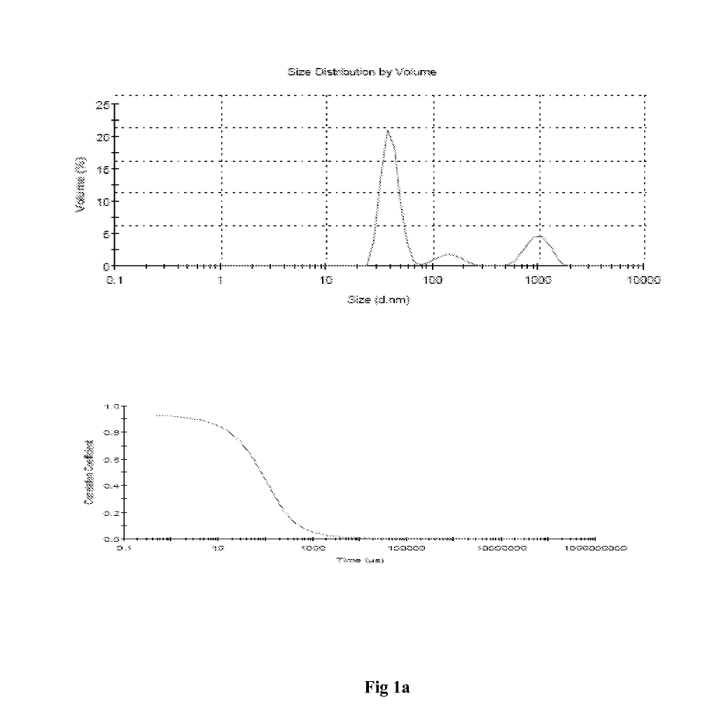

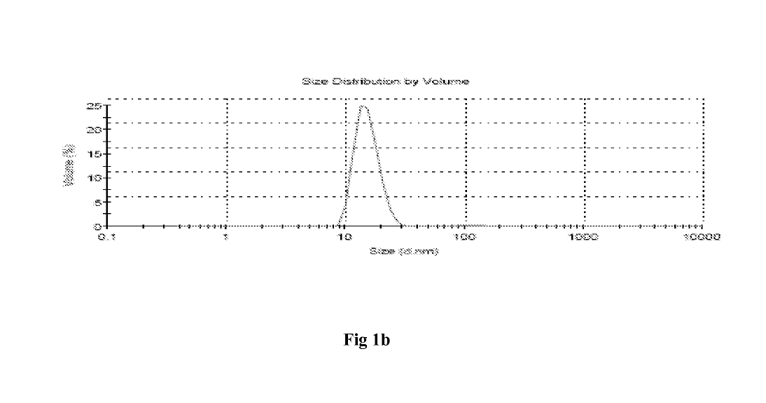

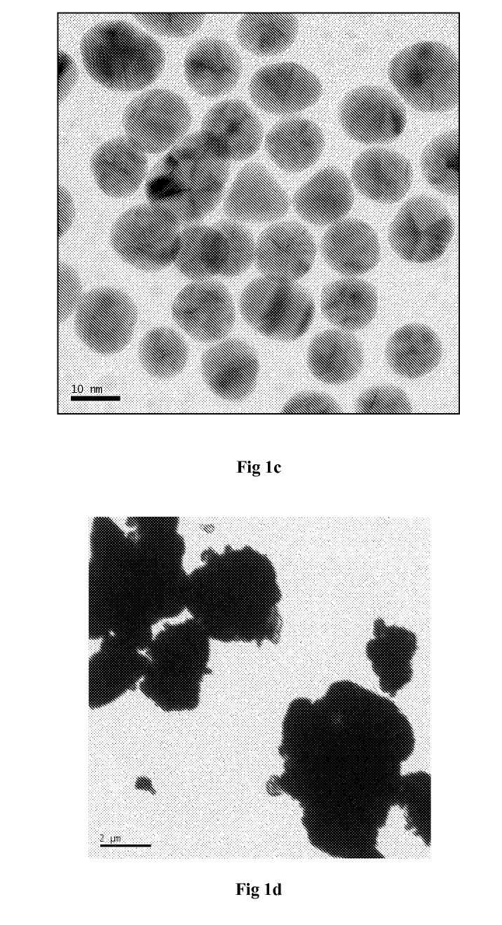

The size analysis data showed that 70.9% of particles in SB preparation had a diameter of about 41.1 nm, and 99.1% of particles in the GNP preparation had a diameter of 15.59 nm (Table 2 a & b; FIG. 1a & b).

TABLE 2a
Size distribution report by volume for SB as analyzed
by dynamic light scattering studies. The hydrodynamic
diameter of the particles are given in nanometers (nm).

| | | Diam. (nm) | % Volume | Width (nm) |
|---|---|---|---|---|
| Z-Average (d. nm): | Peak 1: | 41.10 | 70.9 | 8.058 |
| 83.23 | | | | |
| Pdl: 0.547 | Peak 2: | 148.2 | 8.5 | 37.37 |
| Intercept: 0.959 | Peak 3: | 1029 | 20.6 | 248.4 |

TABLE 2b
Size distribution report by volume for GNP as analyzed
by dynamic light scattering studies. The hydrodynamic diameter
of the particles are given in nanometers (nm).

| | | Diam. (nm) | % Volume | Width (nm) |
|---|---|---|---|---|
| Z-Average (d. nm): | Peak 1: | 15.59 | 99.1 | 3.520 |
| 29.72 | | | | |
| Pdl: 0.583 | Peak 2: | 133.3 | 0.7 | 45.56 |
| Intercept: 0.866 | Peak 3: | 421.0 | 0.3 | 108.1 |

The electron microscopy picture of GNP and SB used in the present invention is provided in FIG. 1c and 1d respectively.

The size and the trace element analyses of the different SB samples tested (SB1, SB2 and SB3) showed consistency in 1) the sample composition and 2) the average particle size across the samples. The gold particle size in SB may have various sizes and can be used to perpetuate stemness of stem cells. Particularly, in the instant invention, gold particles of 10-200 nm size in SB are used.

### Example 4

Determination of the dose of SB and GNP: The dose of SB and GNP were determined by studying their cytotoxicity over a range of doses on hESCs. Cytotoxicity was evaluated employing a MTT assay. The control hESCs were grown on MEF in the absence of SB/GNP. The treated groups were exposed to SB or GNP at various concentrations on a 24 well plate for 4 days, in normal ESC media described above. 20 μl of MTT labeling reagent (final concentration 0.5 mg/ml) was added to each of the well. The cells were then incubated in dark for 4 h at 37° C. 200 μl of DMSO was added to each well and mixed well till the color developed. Colored formazan products were quantified by measuring absorbance using an ELISA reader at 550 nm. This experiment was carried out in triplicates.

SB was supplemented to the cultures, for 96 hrs, from day 1 onwards, at concentrations of 0.1, 1, and 5 μg/ml (FIG. 2a). Similarly, the GNP was tested for cytotoxicity at doses of 1, 5, 10 and 20 μg/ml (FIG. 2b). The doses which did not show any cytotoxicity were selected and studied for their effect on hESC proliferation or expansion. The doses of SB and GNP which showed maximum proliferation were selected for subsequent studies.

Doses of 0.1, 1 and 5 μg/ml of SB preparation were tested for its cytotoxic impact on HuES9, grown in normal hESC media, by MTT assay. The doses of 0.1 and 1 μg/ml SB particles did not show any cytotoxicity. However, 5 μg/ml dose showed upto 11.3% cytotoxicity. Concentrations of 0.1 and 1 μg/ml SB were therefore considered as the safe doses for hESC culture supplementation and were screened further to evaluate the effect on proliferation and pluripotency of the hESC lines (FIG. 2a).

To see if gold alone cause cytotoxicity and alter proliferation and pluripotency of ESCs, the GNPs were tested for their cytotoxicity. The GNPs were screened for at 1, 5, 10, 15 and 20 μg/ml concentrations on the same hESC line. The GNP which is also known to be inert exerted no cytotoxicity up to 15 μg/ml concentrations. The GNPs showed no cytotoxicity till a dose of 20 μg/ml. However, 25 μg/ml dose showed upto 15.4% cytotoxicity. Doses of 1 and 10 μg/ml GNP were further tested for effect on proliferation and pluripotency of hESCs (FIG. 2b).

### Example 5

Determination of the effect of gold particles on stemness: To determine the effect of SB and GNP on the proliferation/self renewal and pluripotency of hESCs, the hESC line (HUES9) was maintained in FGF2 supplemented ES media on MEFs, on 35 mm culture dishes (BD Falcon). The culture dishes were divided into groups of (1) control: without SB and GNP (hES-C+F, n=4), (2) with supplementation of SB (hES-SB+F, n=4) and (3) with supplementation of GNP (hES-GNP+F, n=4) using the selected noncytotoxic doses after a MTT assay. Whether the effect of the gold particles (SB/GNP) on the improvement of hESC pluripotency and proliferation was due to gold alone or if it was a synergistic effect of gold with FGF2 was also studied by culturing cells in absence of FGF2. hESCs were cultured on 35 mm dishes (BD Falcon) divided into multiple groups of control without FGF2 (hES-C, n=4) and SB without FGF2 (hES-SB, n=4) and GNP without FGF2 (hES-GNP, n=4). The effect on pluripotency was determined by quantitative (qPCR) mRNA expression of the pluripotency marker genes like ABCG2, Nanog and Oct4. The cell proliferation analyses was carried out using the doses of SB and GNP which induced maximum mRNA expression of the pluripotency markers like ABCG2 on day 5.

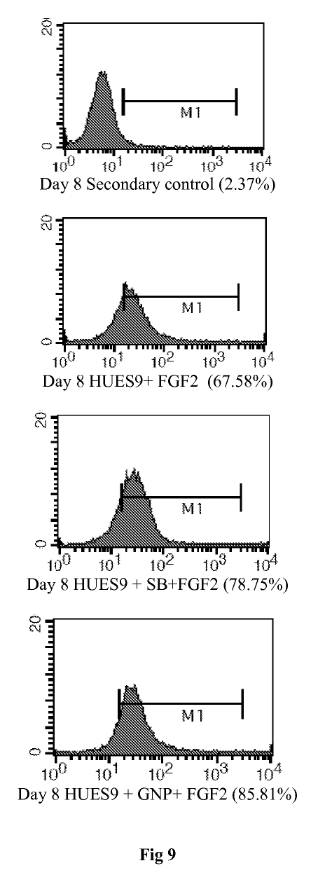

Flow cytometry (FACS) analyses on hESCs for estimating the percentage of pluripotent cells: The effect of the gold particulate preparations on the pluripotency of hESC was studied by culturing the hESCs on MEF coated 35 mm culture dishes in several groups. The control was without SB or GNP supplementation. The treated groups were supplemented with SB or the GNP+FGF2. The cells were harvested from each group on day 8 of culture. The cells were stained with pluripotency surface marker Tra 1-60 antibody and the level of expression of Tra 1-60 in different groups was determined by FACS analyses (FIG. 9). The control groups grown in presence of FGF2 alone showed pluripotency in 67.58% cells, however, the groups treated with SB+FGF2 showed pluripotency in 78.75% cells, and the groups treated with GNP+FGF2 showed pluripotency in 85.81% cells.

### Example 6

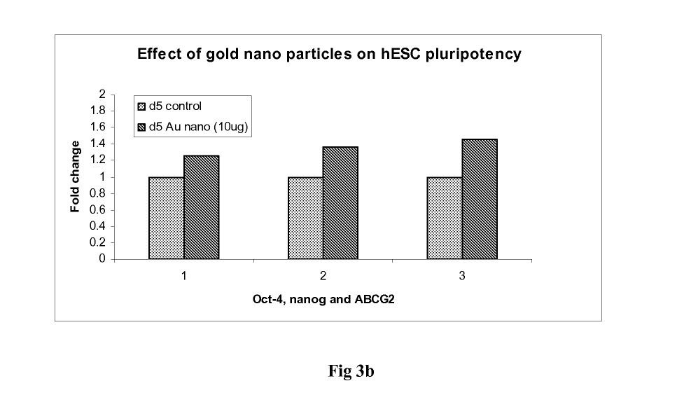

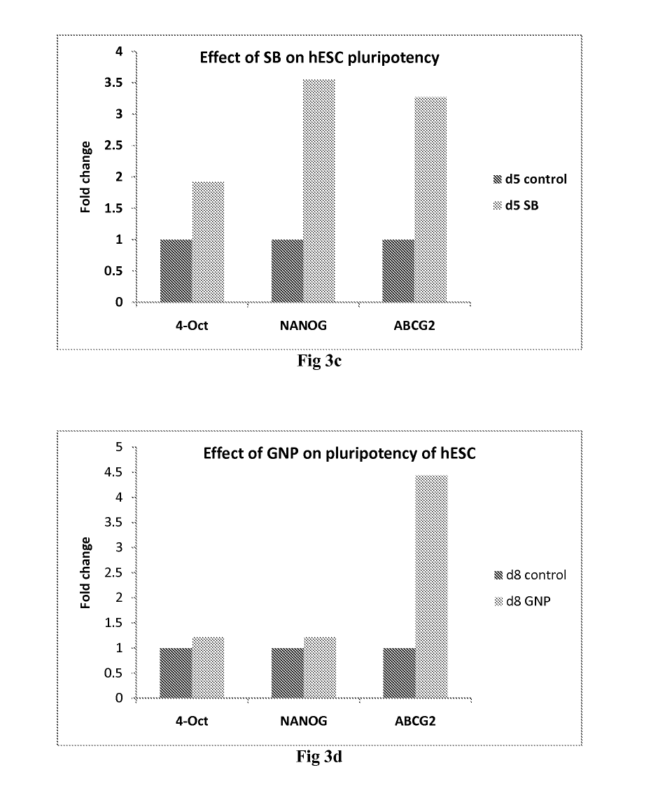

Identification of the dose of SB and GNP that improves pluripotency: Since 0.1 and 1 μg/ml SB were found to be non toxic to the HUES9 cells, the ability of these doses to enhance the pluripotency was investigated by comparing the mRNA expression levels of the pluripotency marker ABCG2. The level of ABCG2 expression in the treated groups hES-SB+F was compared to the control group (hES-C+F). It was found that the hES-SB+F group supplemented with 1 μg/ml SB expressed almost 10 folds more ABCG2 than the control on day 8 (FIG. 3a). This dose of SB was selected for further evaluation on improvement of “stemness” of the ESCs. To determine if 1 μg/ml of SB supplementation alone could improve the pluripotency without FGF-2; the expression levels of ABCG on day 8 in the group hES-SB (hESCs maintained without FGF-2) was compared to that of the hES-SB+F groups. It was found that there was fall in the level of ABCG2 expression of about 477 folds in absence of FGF2 supplementation. However, this change in relative gene expression compared to the normal control group (hES-C+F) was observed with no SB supplementation.

Similarly, the dose of GNP which improves hESC pluripotency was determined by comparing the expression of ABCG2 in the control (hES-C+F) with that of the 1 and 10 μg/ml GNP treated (hES-GNP+F) groups. It was found that the expression of ABCG2 increased by 1.5 fold in the cells supplemented with 10 μg/ml GNP and FGF2 (hES-GNP+F), as compared to the control, as early as on day 5 (FIG. 3b). Though the groups treated with 1 μg/ml GNP (hES-GNP+F) showed expression of ABCG2, it was less as compared to 10 μg/ml GNP. The dose of 1 μg/ml GNP was therefore selected as the dose for further evaluation of the functions of GNPs. In the next step, it was verified if this dose of GNPs alone (i.e., without FGF2 supplementation) could induce the upregulation of ABCG2 expression. QRT-PCR analyses of ABCG2 expression in the cells treated with 10 μg/ml GNP without FGF2 (hES-GNP) showed a 1.5 fold fall in its level as compared to the hES-GNP+F group. Beside ABCG2, the 1 μg/ml dose of GNP with FGF-2 supplementation showed 1.3 and 1.4 fold increase in the expression of Oct4 and Nanog respectively in the HuES9 cells on day 5 (FIG. 3b). Similarly, the expression of Oct4, Nanog and ABCG2 mRNA was expressed at significantly higher levels in the SB+FGF2 treated samples as compared to the control with FGF2 alone (FIG. 3c). Quantitative analyses of the mRNA expression levels of pluripotency markers Oct4, Nanog, ABCG2 on day 8 was tested in the control and GNP+FGF2 treated samples. The levels of pluripotency marker expression were significantly higher in the GNP+FGF2 treated samples (FIG. 3d) as compared to the control. These data indicate that the expression of pluripotency markers was higher in the SB or GNP+FGF2 treated samples as compared to the control on both day 5 and day 8.

### Example 7

Effect of SB and GNP particles on spontaneous differentiation: The effect of the gold particulate preparations on the spontaneous differentiation of hESC was studied by culturing the ESCs on MEF coated 35 mm culture dishes in several groups. The control (n=9) were without SB or GNP supplementation. The treated groups were supplemented with SB (n=9 plates) or the GNP (n=9). The cells were harvested (n=3 each) from each group on days 5, 8, and 11 of culture. The RNA was isolated and the expression of pluripotency (Oct-4, Nanog), ectoderm (Nestin, βIII tubulin, endoderm (GATA-4, AFP) and mesoderm (Brachury, BMP-2) were tested.

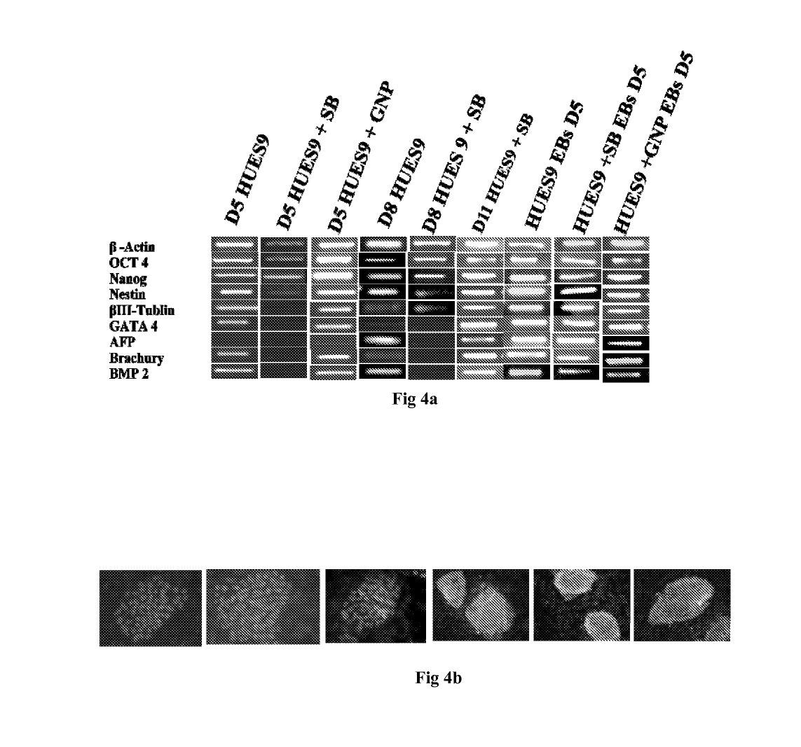

The doses of SB (1 μg/ml) and GNP (10 μg/ml) which enhanced the expression of the pluripotency gene ABCG2 was tested for their ability to reduce spontaneous differentiation. The hESC (HUES9) cultures were tested for the expression of markers associated with pluripotency and ectoderm, endoderm and mesoderm lineages by RT-PCR. HUES9 were grown in the continuous presence and absence of SB for 5 passages. On the sixth passage cells were harvested on day 5 and day 8. The pluripotency and lineage marker expression in these samples were checked by RT PCR. We found that pluripotency markers OCT4 and Nanog were expressed in all the samples on both day 5 and 8. Basal expression of most of the early lineage markers including ectoderm, endoderm and mesoderm such as βIII-tubulin, nestin, GATA-4, AFP, BMP-2 and Brachyury were found to be positive on day 5 and day 8 HUES9 control (FIG. 4a). However, in the SB treated group, all the lineage markers except the ectoderm markers were found to be silenced on both day 5 and day 8 (FIG. 4a). Immunocytochemistry was performed to confirm the protein expression and localization of pluripotency markers Oct-4 (nuclear) and SSEA4 (surface). Oct-4 and SSEA4 expressions were found both in the control and SB treated day 5 HUES9 colonies (FIG. 4b). Since expression of lineage markers were found down-regulated in the HUES9 cultures exposed to SB, till day 8, we also screened for the lineage expressions in these cultures on day 11. We found that the cells started showing expression of all the three early germ layer lineage markers by this day of culture. These findings indicated a reduction in spontaneous differentiation and maintenance of pluripotency in the hESCs following SB supplementation.

To determine the effect of GNP on spontaneous differentiation, HUES9 cells were grown with or without 10 ug/ml GNP supplementation to normal hESC culture medium. This dose of GNP was found to upregulate the mRNA expression of the pluripotency markers ABCG2, Oct-4 and Nanog on day 5. Since SB down-regulated the induction of early lineage markers till day 8, we checked the expression profile of pluripotency and lineage markers in hESC exposed to GNP on day 5. RT-PCR analyses indicated a positive expression for all the pluripotency and early lineage markers (Oct-4, Nanog, βIII-tubulin, Nestin, GATA-4, AFP, BMP-2 and Brachyury) in both the control and gold nanoparticles treated HUES9 cells (FIG. 4a). Immunocytochemistry was performed to confirm the protein expression and localization of pluripotency markers Oct-4 (nuclear) and SSEA4 (surface). Oct-4 and SSEA4 expressions were found both in the control and GNP treated day 5 HUES9 colonies (FIG. 4b).

### Example 8

Cell expansion/proliferation assay: The effect of SB and GNP on the proliferation of hESC was studied on the HUES9 cells. The hESCs were split in equal seeding densities in groups of control and the treated containing the selected doses of SB (1 μg/ml) and GNP (10 μg/ml). The total number of cells per plate was counted on day 4, 6, and 8. For this, the cells were harvested by trypsinizing the plates (n=3) for each group and the cell expansion was calculated by counting the number of cells/plate using a haemocytometer.

The control plates had an average of 2.83±0.12×10⁵, 6.1±0.7×10⁵ and 7.95±0.9×10⁵ cells/plate on days 4, 6 and 8 respectively. The SB treated plates showed increased counts of 4.08±0.3×10⁵, 7.08±0.8×10⁵ and 8.54±0.7×10⁵ cells/plate on days 4, 6 and 8 respectively; the GNP treated plates showed a count of 3.5×10⁵, 7.3×10⁵ and 9.21×10⁵ cells/plate on an average on days 4, 6 and 8 respectively (FIG. 5).

### Example 9

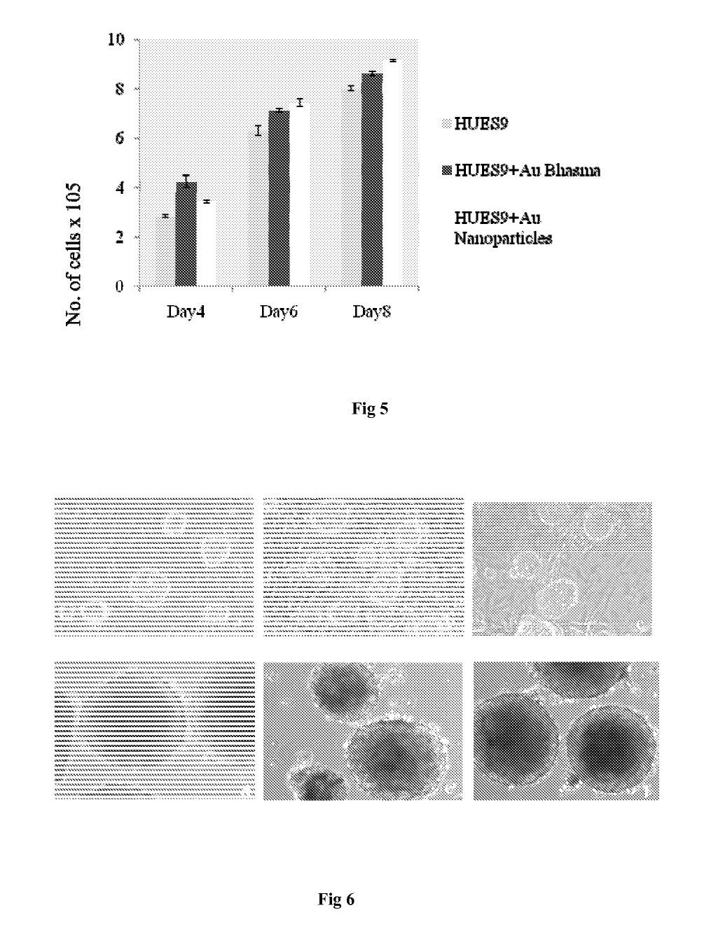

Induction of Embryoid bodies (EBs): Following the determination of doses of SB and GNP which improved proliferation and expression of pluripotency markers, it was verified if the particles had any adverse consequence on the ability of the hESCs to differentiate into the three germ lineages. EBs were grown in suspension culture by mechanical cutting of day 4 control hESC and hESC colonies grown in presence of gold particles (SB or GNP) and seeding them into 60 mm low-adherent dishes (BD Falcon) containing ES media without FGF2. The EBs formed from both the untreated and treated HUES9 cells appeared morphologically normal under the microscope (FIG. 6). After 48 hours, EBs with round morphology were picked up under a stereo zoom binocular microscope with understage illumination (Nikon Stereo microscope SMZ 1500) and transferred to fresh dish and supplemented with fresh media. Day 5 old EBs was then analyzed for the expression of pluripotency and lineage markers like Oct-4, Nanog, βIII-tubulin, Nestin, GATA-4, AFP, BMP-2 and Brachyury. All the pluripotency and lineage markers were expressed in day 5 control, SB and GNP treated groups (FIG. 4a RT-PCR data).

### Example 10

Indirect Immunofluorescence: Day 4 hESCs grown on cover slips coated with MEFs were fixed with 4% Para formaldehyde, followed by permiablization with 0.1% TritonX 100 (Sigma) Protein expression of the pluripotency marker Oct-4 and ES cell marker FGF-R1 were analyzed by incubating the plates with primary anti-Oct-4 immunoglobulin-IgG (R &D Systems Inc., MD, USA) overnight at 4° C. After washing thrice with PBS, FITC conjugated secondary antibodies against primary antibodies were added and incubated for 2 hours at room temperature. Slides were mounted with vectashield mounting medium containing DAPI (Vector Laboratories, Inc. Burlingame, Calif.). Negative control slides were incubated only with the secondary antibodies. Images were acquired using Nikon Eclipse 80i microscope (Nikon Corporation, Japan) and Q capture Pro 6 software (Q Imaging Corporation, BC, Canada). The results of image are provided in FIG. 6.

### Example 11

RNA isolation and RT-PCR: Total RNA from the various hESC samples and EBs was isolated using TRIZOL-LS reagent (Invitrogen) as per the manufacturer's protocol. Complementary DNA was synthesized using the Superscript III First-Strand Synthesis System (Invitrogen) from 1 μg of total RNA in a reaction volume of 20 μL as per the manufacture's directions. PCR was carried out using 1 U Tag DNA polymerase (Sigma) and MgCl2 to a final concentration of 1.5 mM in a total volume of 25ul/reaction. β-Actin was used as the housekeeping control. PCR cycles consisted of an initial denaturation at 94° C. for 45 s and extension at 72° C. for 10 min. Primers for puripotency (Oct4, Nanog, ABCG2), ectoderm (βIII-tubulin, Nestin), endoderm (Gata4, AFP) and mesoderm (BMP2, _Brachury_) were screened. The RT-PCR primer sequences, annealing temperatures and the amplicon sizes are listed in Table 3.

| | | Annealing | Pro- |
|---|---|---|---|
| | | Temper- | duct |
| | | ature | Size |
| Gene | Sequence | (° C.) | (bp) |
| β-Actin | GCTCGTCGTCGACA ACGGCTC | 54 | 353 |
| | CAAACATGATCTGGGTCATCTTCTC | | |
| Oct 4 | CGACCATCTGCCGCTTTGAG | 57 | 572 |
| | CCCCCTGTCCCCCATTCCTA | | |
| Nanog | CCTCCTCCATGGATCTGCTTATTCA | 57 | 262 |
| | CAGGTCTTCACCTGTTTGTAG | | |
| Nestin | AACAGCGACGGAGGTCTCTA | 55 | 220 |
| | TTCTCTTGTCCCGCAGACTT | | |
| βIIITubulin | CTTGGGGCCCTGGGCCTCCGA | 60 | 174 |
| | GGCTTCCTGCAGTGGTACACGGGCG | | |
| GATA4 | TCCAAACCAGAAAACGGAAG | 60 | 187 |
| | CTGTGCCCGTAGTGAGATGA | | |
| AFP | AGAACCTGTCACAAGCTGTG | 60 | 576 |
| | GACAGCAAGCTGAGGATGTC | | |
| Brachury | ACCCAGTTCATAGCGGTGAC | 60 | 216 |
| | ATGAGGATTTGCAGGTGGAC | | |
| BMP2 | TGTATCGCAGGCACTCAGGTCAG | 60 | 328 |
| | AAGTCTGGTCACGGGGAAT | | |
| FGFR1 | GGACTCTCCCATCACTCTGCAT | 56 | 109 |
| | CCCCTGTGCAATAGATGATGATC | | |
| IGF2 | CAATGGGGAAGTCGATGCTG | 61 | 421 |
| | CTTGGCGAGCACGTGAC | | |

To study if the SB or GNP particles improved pluripotency by increasing the niche population, the hESC colonies for increase in percentage of the FGFR1 positive niche cells/colony and for mRNA expression of niche specific FGFR1 and IGF2 were analyzed. Primers for IGF2 and FGFR1 were checked for RT PCR amplification of the various samples. The PCR products were electrophoresed on 1.5% agarose gels, The expression level of the FGFR1 and IGF2 genes was evaluated by ImageJ software from the bands obtained (http://rsb.info.nih.gov/ij), and the areas under the curves were calculated and analyzed. Expression levels of FGFR1 and IGF2 was quantified relative to β-actin expression level and expressed as arbitrary units.

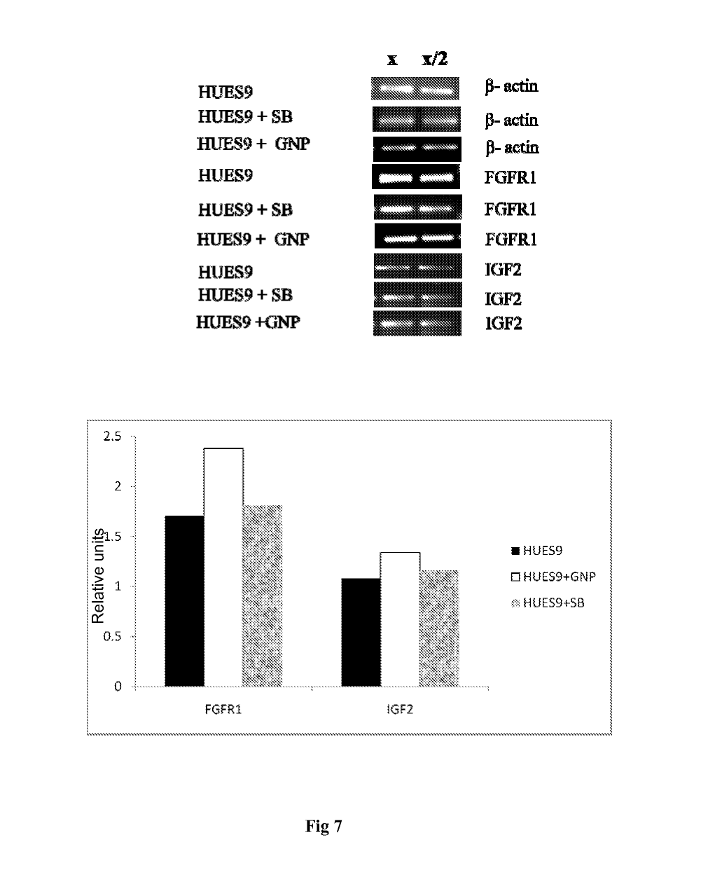

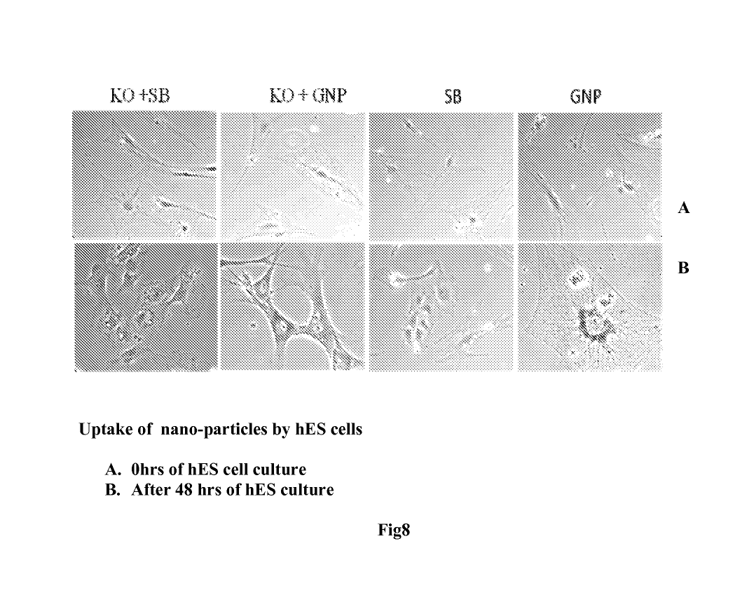

The relative level of expressions of FGFR1 and IGF2 in day 8 HUES9 cells grown in the presence of SB and GNP was determined by RT-PCR. IGF2 and FGFR expression was seen to be enhanced by 1.16 and 1.81 (relative units) respectively in SB treated cells, 1.34 and 3.38 (relative units) respectively in GNP treated cells, as compared to the control which showed IGF2 and FGFR expression level of 1.08 and 1.71 (relative units) respectively (FIG. 7).

### Example 12

Long term culture of hESCs in presence of SB and GNP has no adverse effect on pluripotency, karyotype and freeze-thaw efficiencies: HUES9 cells were maintained in continuous presence of SB and GNP for 15 passages. Cytogenetic analysis performed on twenty five G-banded metaphase cells from HUES9 cells demonstrated no karyotypic alterations at the end of the 15 passages. The HUES9 cells were also collected to analyze for the expression of pluripotency and markers of ectoderm, endoderm and mesoderm lineages. We found that the hESCs retained strong expression of Oct-4, and Nanog; To confirm if the presence of SB or GNP impacted the pluripotency of the cells, HUES9 cells cultured in presence of these particles upto 15^{th} passage were harvested and induced for EB formation. Further, the EBs were collected on day 5 and analyzed for successful formation of early progenies using RT-PCR analyses for the expression of ectoderm, endoderm and mesoderm lineage markers (FIG. 4a).

Quantitative (q) RT-PCR: Pre-designed Assays on Demand TaqMan probes and primers were procured from Applied BioSystems. Total RNA was extracted from undifferentiated hES cells and EBs and reverse transcribed using Superscript II (Invitrogen). qRT-PCR analysis was conducted using ABI PRISM 7500 Sequence Detection System (Applied BioSystems). After an initial denaturation for 10 mins at 95° C., the reaction was run for 40 cycles of PCR (95° C. for 15 sec, 60° C. for 1 min). Changes in gene expression (in triplicates) were normalized to 18S rRNA levels in terms of fold change.

Karyotyping: The karyotype analysis of HuES9 grown in the absence and presence of SB and GNP was done at passage 10 and passage 15. HuES9 cells in the exponential growth stage were treated for 2 hours with colcemid (0.1 μg/ml). After colcemid treatment, digested single cells were karyotyped using G-banding method. The karyotype of HuES9 cells treated with SB and GNP was found to be normal (representative pattern observed as in FIG. 2c).

What is claimed is:

1. Gold nanoparticle (GNP) at concentration of about 0.1 μg/ml to about 20 μg/ml for perpetuating stemness of stem cells.

2. The GNP as claimed in claim 1, wherein the concentration of GNP is preferably about 10 m/ml and diameter is about 15 nm to about 16.5 nm.

3. The GNP as claimed in claim 1, wherein the stem cells are lineage-uncommitted pluripotent stem cells.

4. A method to perpetuate stemness of stem cells comprising step of growing the stem cells in presence of gold particle or Swarna Bhasma.

5. The method as claimed in claim 4, wherein the method perpetuates stemness by enhancing proliferation, self-renewal and pluripotency and reducing spontaneous differentiation of the stem cells.

6. The method as claimed in claim 4, wherein the gold particle is gold nanoparticle (GNP) present at a concentration of about 0.1 μg/ml to about 20 μg/ml, preferably about 10 μg/ml; or wherein the Swarna bhasma is present at a concentration of about 0.11 μg/ml to about 2 μg/ml, preferably about 1 μg/ml.

7. The method as claimed in claim 4, wherein the stem cells are lineage-uncommitted pluripotent stem cells.

8. A composition for enhancing pluripotency of stem cells, said composition comprising Fibroblast Growth Factor along with gold particles or Swarna Bhasma.

9. The composition as claimed in claim 8, wherein the gold particle is gold nanoparticle (GNP) present at a concentration of about 0.1 μg/ml to about 20 μg/ml, preferably about 10 μg/ml and having diameter of about 15 nm to about 16.5 nm; or wherein the Swarna bhasma is present at a concentration of about 0.1 μg/ml to about 2 μg/ml, preferably about 1 μg/ml.

10. The composition as claimed in claim 8, wherein the Fibroblast Growth Factor is a basic fibroblast growth factor or FGF2.

11. The composition as claimed in claim 10, wherein the FGF2 is present at a concentration of about 2 ng/ml to about 40 ng/ml, preferably about 4 ng/ml.

12. The composition as claimed in claim 8, wherein the stem cells are lineage-uncommitted pluripotent stem cells.

13. A medium for perpetuating stemness of stem cells, said medium comprising components of ES media along with gold particle at a concentration of about 0.1 μg/ml to about 25 μg/ml or Swarna Bhasma at a concentration of about 0.1 μg/ml to about 2 μg/ml.

14. The medium as claimed in claim 13, wherein the gold particle is gold nanoparticle (GNP) having concentration preferably of about 10 μg/ml and having diameter of about 15 nm to about 16.5 nm, preferably about 15.59 nm; or wherein the concentration of the Swarna bhasma is preferably about 1 μg/ml.

15. The medium as claimed in claim 13, wherein the stem cells are lineage-uncommitted pluripotent stem cells.

16. The medium as claimed in claim 13, wherein the components of ES media include 80% DMEM, 20% Knockout serum replacer, L-glutamine at concentration of about 2 mM, 1% nonessential aminoacids, β-mercaptoethanol at concentration of about 0.1 mM, human FGF2 at concentration of about 4 ng/ml and penicillin streptomycin at concentration of about 50 U/ml.

17. A composition for perpetuating stemness of stem cells, said composition comprising Swarna Bhasma and gold particles, preferably gold nanoparticles.
